(19) United States
(12) Patent Application Publication
(10) Pub. No.: US 2010/0286618 A1
(43) Pub. Date: Nov. 11, 2010

(54) MULTI NEEDLE

(76) Inventor: Jong-Soo Choi, Seoul (KR)

Correspondence Address: HARNESS, DICKEY & PIERCE, P.L.C., P.O. BOX 8910, RESTON, VA, 20195, US

(21) Appl. No.: 12/812,117

(22) PCT Filed: Mar. 7, 2008
(86) PCT No.: PCT/KR08/01304

§ 371 Date: Jul. 8, 2010

(30) Foreign Application Priority Data
Jan. 14, 2008 (KR) 10-2008-0004010

(51) Int. Cl. A61M 5/32

(52) U.S. Cl. 604/173

(57) ABSTRACT

An injector and a needle, which is engaged to the injector by using a needle hub, are disclosed. The multi needle comprises a needle hub which consists of one inlet into which a nozzle of an injector is inserted, and a plurality of outlets communicating with the inlet; and a plurality of needles which are inserted into the outlets of the needle hub, respectively.

### TECHNICAL FIELD

The present invention relates to an injector and a needle, which is engaged to the injector by using a needle hub.

### BACKGROUND ART

Generally, an injector includes a cylinder and a piston inserted into the interior of the cylinder. Injection liquid is stored in the interior of the cylinder. A nozzle is provided at an end of the cylinder for discharging injection liquid. The piston is inserted into the cylinder with a packing being engaged at its front end for pressurizing in a direction of the nozzle. A pressurizing member is provided at its rear end for pressing the piston.

When injection liquid is injected into a human being body or an animal, a needle connected to the injector is needed. As shown in FIG. 1, the needle 10 is sold in a state that it is engaged to the needle hub 20 or is sold separately from the same or is sold along with the injector. When injection liquid L is injected into a human being body or an animal, as shown in FIG. 1, the needle hub 20 having the needle 10 is inserted into the nozzle 31 of the injector 30.

In recent years, a beauty business and plastic surgery become popular. So, a surgical operation using an injector increases day by day in a beauty business and plastic surgery. As one representative example, there is a surgical operation that a beauty liquid like a filler or toxin is injected into a skin. In particular, when injecting injection liquid, the injection liquid is injected into both a dermis and a subcutaneous layer, and it is needed to uniformly inject a beauty injection liquid into a target portion of skin. During an injection operation, a needle of the injector is inserted into a skin of a person, and a certain amount of injection liquid is injected, and the needle is removed, and then is inserted to another portion of the skin. However, the above repeated injection operation takes a lot of time while causing many inconveniences in work. Since injection operations are repeatedly performed, it is needed to insert the needles multiple times by certain uniform depths. In this case, a doctor needs an intensive attention and professional skill. When the insertion depths of needles are different in different portions of the skin, it is impossible to achieve a desired effect of the injection liquid since the injection liquid is not uniformly injected into the skin.

### DISCLOSURE OF INVENTION

Accordingly, it is an object of the present invention to provide a multi needle which overcomes the problems encountered in the conventional art.

To achieve the above object, there is provided a multi needle which comprises a needle hub which consists of one inlet into which a nozzle of an injector is inserted, and a plurality of outlets communicating with the inlet; and a plurality of needles which are inserted into the outlets of the needle hub, respectively.

According to one embodiment of the present invention, a plurality of the outlets and a plurality of the needles are arranged in one row, respectively.

According to one embodiment of the present invention, a plurality of the outlets and a plurality of needles are arranged in multiple rows and columns.

According to one embodiment of the present invention, there is further provided an adjusting member which is extended from the needle hub so that a tip of each needle can be exposed while covering an intermediate portion of each needle, respectively.

According to one embodiment of the present invention, there is further provided an adjusting member which is inserted into the needle hub and surrounds an intermediate portion of each needle so that a tip of each needle can be exposed, respectively.

According to one embodiment of the present invention, the needle hub comprises a first body which is connected with the nozzle of the injector with the inlet being formed at an end of the first body, and with an accommodating space, which communicates with the inlet, being formed at the other end of the first body; and a second body through which a plurality of the outlets pass with the second body being inserted into the accommodating space so that each outlet can communicate with the inlet, respectively.

According to one embodiment of the present invention, second body is longitudinally formed so that a tip of each needle being exposed, and with an intermediate of each needle being covered, respectively.

According to one embodiment of the present invention, there is further provided a protection cap which is inserted into the needle hub for surrounding the needle so as to protect the same while not exposing the tip of each needle to the outside.

### BRIEF DESCRIPTION OF THE DRAWINGS

The present invention will become better understood with reference to the accompanying drawings which are given only by way of illustration and thus are not limitative of the present invention, wherein;

FIG. 1 is a schematic view illustrating an operation that a skin is injected by using a conventional injector and a needle;

FIG. 2 is a perspective view illustrating a multi needle and an injector according to an embodiment of the present invention;

FIG. 3 is a cross sectional view illustrating a state that a multi needle of FIG. 2 is disassembled;

FIG. 4 is a cross sectional view illustrating a construction that a multi needle of FIG. 2 is engaged to an injector;

FIG. 5 is a cross sectional view illustrating a multi needle according to another embodiment of the present invention;

FIG. 6 is a cross sectional view illustrating a multi needle according to further another embodiment of the present invention;

FIG. 7 is a perspective view illustrating a multi needle according to still further another embodiment of the present invention;

FIG. 8 is a schematic view illustrating an operation that a skin is injected by using a multi needle according to the present invention; and

FIG. 9 is a perspective view illustrating a multi needle according to still further another embodiment of the present invention.

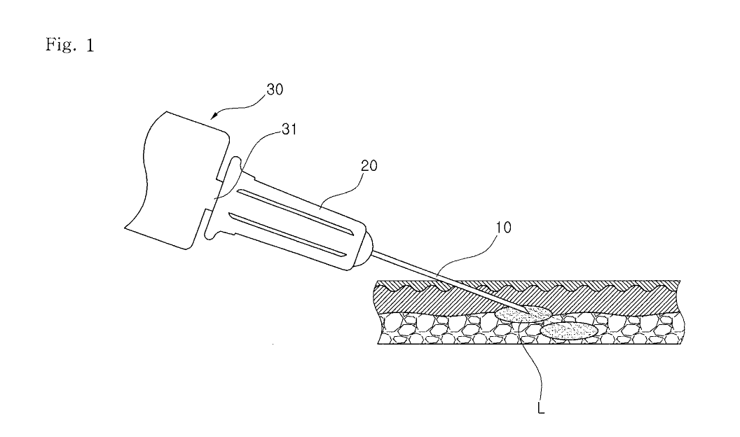

### BEST MODE FOR CARRYING OUT THE INVENTION

The preferred embodiments of the present invention will be described with reference to the accompanying drawings. In the descriptions of the present invention, the same elements are given the same reference names and numerals. The additional description thereto will be omitted.

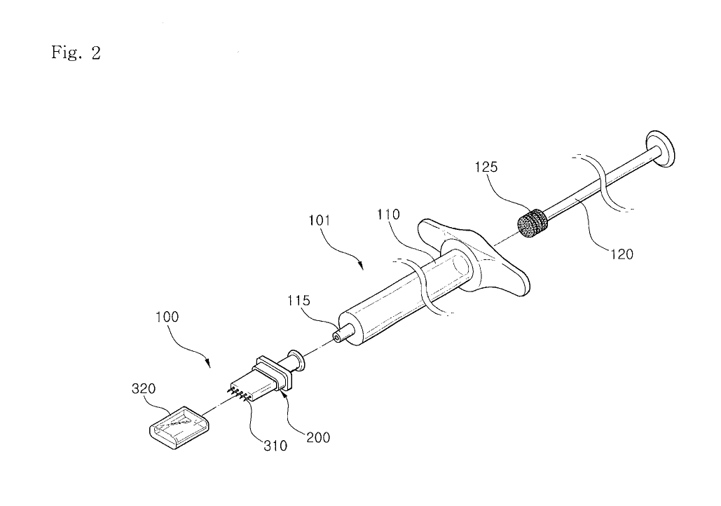

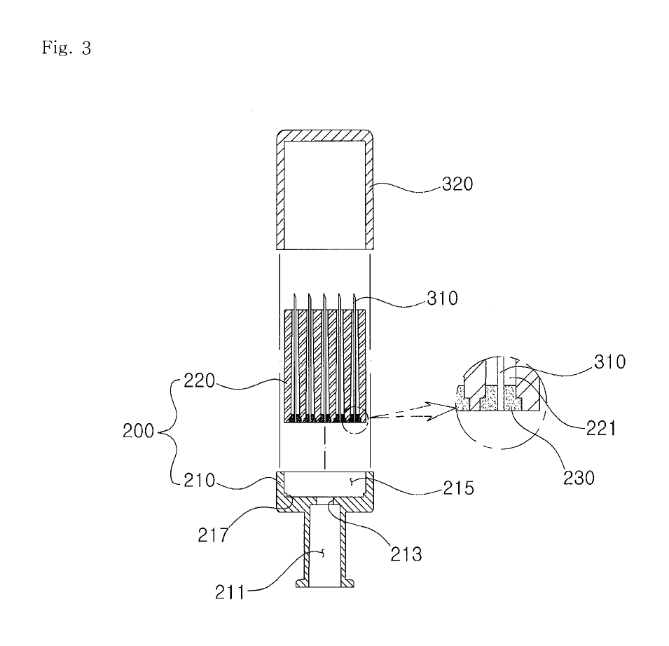

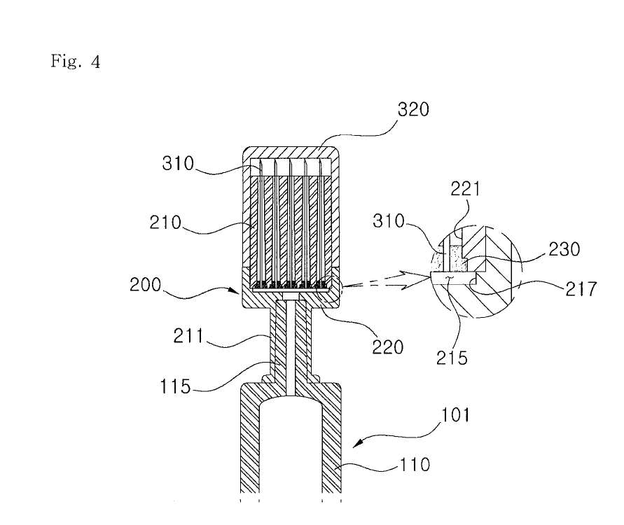

FIG. 2 is a perspective view illustrating a multi needle and an injector according to an embodiment of the present invention, FIG. 3 is a cross sectional view illustrating a state that a multi needle of FIG. 2 is disassembled, and FIG. 4 is a cross sectional view illustrating a construction that a multi needle of FIG. 2 is engaged to an injector.

As shown in FIGS. 2 through 4, a multi needle 100 according to an embodiment of the present invention is connected to an injector 101 when it is used. The injector 101 comprises a piston 120 and a cylinder 110. A nozzle 115 is protruded from a front end of the cylinder 110, and a packing 125 is engaged at a front end of the piston 120.

The multi needle 100 according to an embodiment of the present invention is engaged to a nozzle 115 of the injector 101 when it is used. The multi needle 100 consists of a needle hub 200 engaged at the nozzle 115, and a plurality of needles 310 engaged at the needle hub 200. The needle hub 200 is provided with one inlet 211 and a plurality of outlets 221. As shown in FIGS. 2 through 4, the inlet 211 of the needle hub 200 is protruded, and the nozzle 115 of the injector 101 is inserted into an inner side of the same. The outlets 221 of the needle hub 200 communicate with the inlet 211 at the inner side of the needle hub 200. So, an injection liquid inputted into the interior of the needle hub 200 through the inlet 211 is discharged to the outside of the needle hub 200 through a plurality of the outlets 221. As shown in FIGS. 2 through 4, the needle 310 is provided in multiple numbers, and each needle 310 is inserted into each outlet 221, respectively. Here, the needle 310 may be pressed and fixed at the outlet 221, but as shown in FIGS. 3 and 4, may be fixed as a sealing fixture 230 made of a certain material such as silicon is filled between the outlets 221 and the needle 310.

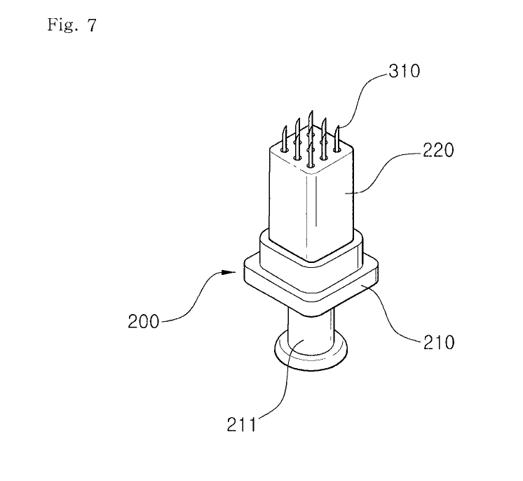

In the needle hub 200 according to an embodiment of the present invention, as shown in FIG. 2, the outlets 221 and the needles 310 may be arranged in a row. So, it is possible to uniformly inject an injection liquid in a relatively wider area of the skin at one time. The above construction is not limited thereto. In another embodiment of the present invention, as shown in FIG. 7, the outlets 221 and the needles 310 may be arranged in multiple rows and columns, so that it is possible to inject wider areas of skin at one time.

As shown in FIGS. 3 and 4, for an easier manufacture of the needle hub 200, the needle hub 200 may be formed of two pieces, namely, a first body 210 and a second body 220.

As shown in FIG. 4, the first body 210 is connected with the injector 101. The inlet 211 is formed at one end of the first body 210, and an accommodating space 215 is formed at the other end of the same. As shown in FIG. 3, the inlet 211 is formed in a protruded cylindrical shape while outwardly protruding from one end of the first body 210, and the accommodating space 215 is formed in a concave groove shape while being grooved from the other end of the first body 210. Here, as shown in FIG. 3, a step 217 is formed at an edge bottom side of the accommodating space 215. The inlet 211 of the first body 210 communicates with the accommodating space 215 through a through hole 213.

A plurality of the outlets 221 pass through the second body 220, with each outlet 221 being engaged with a corresponding needle 310. As described earlier, each needle 310 is pressed and fixed into each outlet 221, respectively, or is may be fixed with the help of the sealing fixture 230. As shown in FIG. 4, the second body 220 is fixed into the first body 210.

As shown in FIG. 4, the second body 220 is fixedly inserted into the accommodating space 215 of the first body 210, while being accommodated in the step 217. A space is formed as much as the volume of the step 217 between the bottom of the accommodating space 215 and the lower side of the second body 220, so that the inlet 211 and the outlets 221 communicate with each other through the space.

As shown in FIGS. 3 and 4, in the multi needle 100 according to an embodiment of the present invention, the needle 310 may be provided with its tip being exposed. For the above construction, the second body 220 is longitudinally extended with an intermediate portion of the needle 310 being covered, and with its tip being exposed. The exposed length of the needle 310 is preferably same as the insertion depth of the needle depending on the purpose of operation. For example, when the multi needle 100 according to the present invention is manufactured for injecting a beauty injection liquid such as toxin and filler, the exposed length of the tip of the needle 310 is preferably determined to be enough to reach a boundary portion of the dermis D and a subcutaneous layer F through an epidermis E.

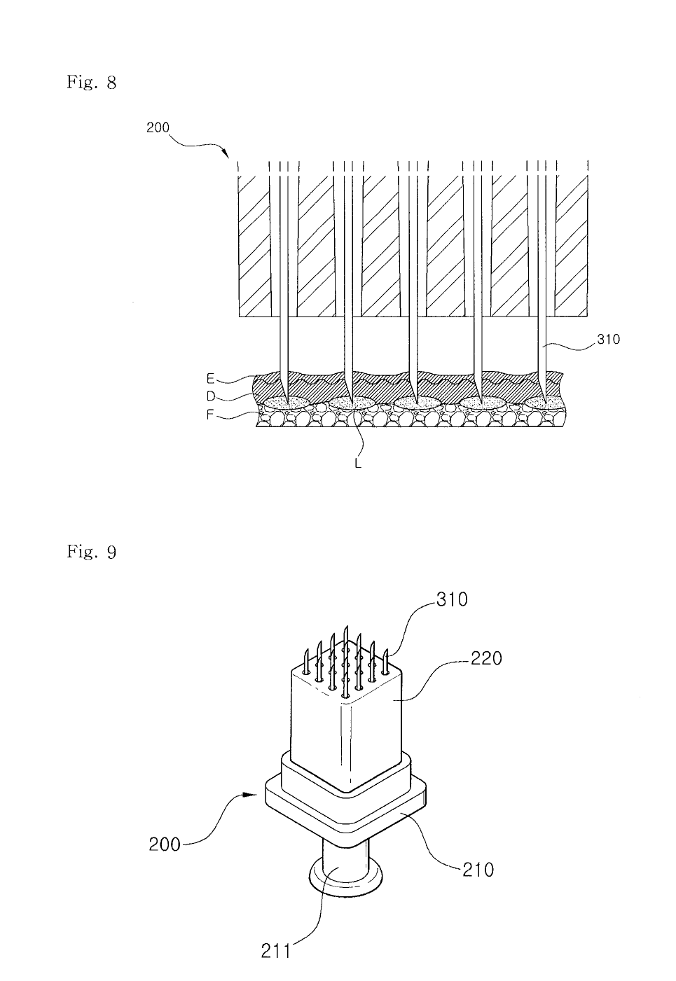

When the needle hub 200 is made with an intermediate portion of the needle 310 being covered, and with its tip being exposed, the insertion depths of the needles 310 can be uniform. As shown in FIG. 8, it is possible to accurately insert each needle 310 up to a desired depth with a simple work that the needle 310 is inserted into the skin just until the end of the needle hub 200 contacts with the skin.

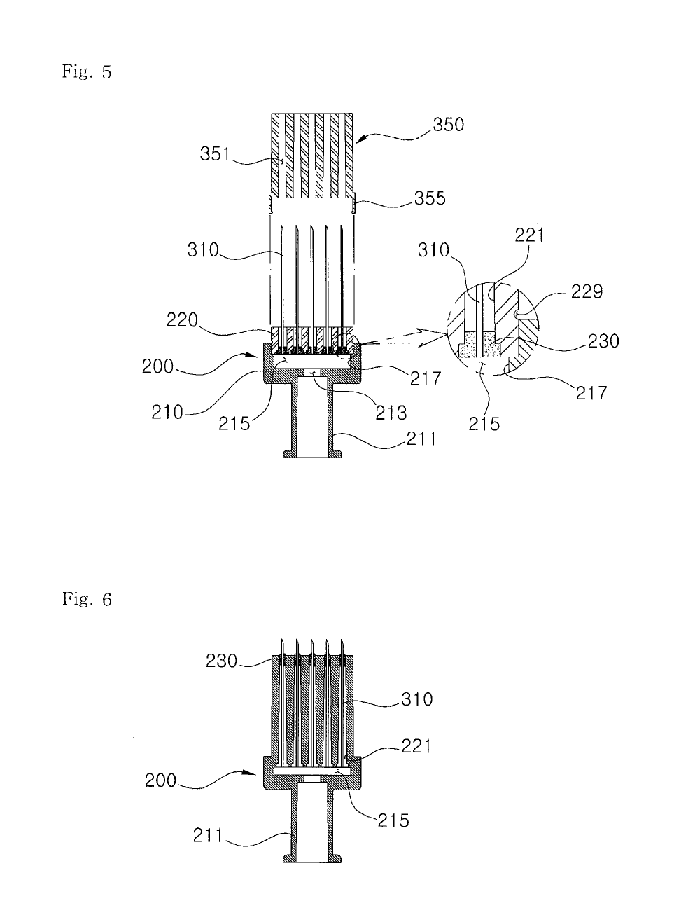

In the multi needle 100 according to the present invention, the second body 220 may be formed in a shorter shape as compared to the earlier described embodiment of the present invention. As shown in FIG. 5, the second body 220 is made shorter and is fixedly inserted into the accommodating space 215 of the first body 210. In this embodiment of the present invention, since the second body 220 is made shorter, the needle 310 may be exposed longer to the outside.

When the needle 310 is formed longer, it is needed to limit the insertion length of the needle into the skin depending on a doctor's selection, and an intensive attention is needed during the operation. So, as shown in FIG. 5, an adjusting member 350 is further provided in the multi needle 100 of the embodiment of FIG. 5 for limiting the insertion depth of the needle 310.

The adjusting member 350 is detachably engaged to the needle hub 200, namely, detachably to the second body 220. For the above construction, a groove 229 is formed on an outer surface of the second body 220, and a hook 355 inserted into the groove 229 is formed at a lower side of the adjusting member 350.

As shown in FIG. 5, a plurality of the adjusting elements 351 are formed in the adjusting member 350. Each adjusting element 351 is arranged opposite to the outlet 221 of the second body 220. When the adjusting member 350 is engaged in the second body 220 of the needle hub 200, each needle 310 arranged in each outlet 221 passes through each adjusting element 351 with a tip of each needle being exposed to the outside. The length of the tip of each needle 310 exposed to the outside through the adjusting member 350 is determined in the same manner as disclosed in the descriptions of FIGS. 4 through 8.

When the adjusting member 350 detachable from the needle hub 200 is provided, the adjusting member 350 may be attached or detached depending on the purpose of operation, and the injection operation can be performed. In another embodiment, a wide range of injection operations having different insertion depths into skins can be performed by varying the lengths of the adjusting members 350 and by varying the exposed lengths of the needles 310.

In the descriptions earlier, the examples that the needle hub 200 of the present invention consists of multiple bodies were explained. The above construction is not limited thereto. In another example, as shown in FIG. 6, the needle hub 200 may be formed integrally.

In this case, as shown in FIG. 5, the adjusting member 350 may be separable from the needle hub 200, but may be integral with the needle hub 200. The needle 310 can be pressed and fixed into the integral needle hub 200 or can be fixed by using the sealing fixture 230.

According to the present invention, the adjusting member 350, which is able to adjust or limit the insertion depth of the needle 310, can be engaged in the needle hub 200. As shown in FIGS. 4 and 6, it can be extended from part of the needle hub 200.

A protection cap 320 may be further provided in the multi needle 100 of the present invention. As shown in FIG. 4, the protection cap 320 is inserted into the needle hub 200 and surrounds and protects the tip of the needle 310. When the protection cap 320 is inserted into the needle hub 200, since the tip of the needle 310 is not exposed to the outside, it is possible to store the needle in safe.

In the multi needle 100 according to the present invention, a plurality of needles 310 may be arranged in a row as shown in FIG. 2 or may be arranged in multiple rows and columns as shown in FIGS. 7 and 8. So, when the injection operation is performed by using the multi needle 100 according to the present invention, as shown in FIG. 8, it is possible to inject a uniform amount of injection liquid into the multiple portions of the skin at one time.

At this time, the insertion depths of the needles 310 are limited by means of the adjusting member 350 or the needle hub 200. So, it is possible to inject an injection liquid into a skin with a uniform injection depth. After a beauty injection liquid is injected into the skin by using the multi needle 100 according to the present invention, the injection is performed to another place as shown in FIG. 8. So, it is possible to perform an accurate injection operation with respect to a relatively wider area of the skin within a shorter time.

### INDUSTRIAL APPLICABILITY

According to the present invention, a plurality of outlets are provided in a needle hub connected to a nozzle of an injector with a needle being provided at each outlet. A plurality of needles can be engaged to one injector. It is possible to inject an injection liquid at a target portion of a skin with a uniform injection depth and a uniform amount of the injection liquid with one time injection. The present invention makes an injection operation simpler and easier as compared to the repeatedly performed injection operations in the conventional art. In addition, the reliability of operation is enhanced.

An adjusting member is provided for exposing a front end of the needle by a certain length. It is possible to reliably inject an injection liquid by inserting a needle into a skin by a desired depth, so a reliable operation is obtained, and a side effect can be significantly reduced.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A multi needle, comprising:
a needle hub which consists of one inlet, into which a nozzle of an injector is inserted, and a plurality of outlets communicating with the inlet; and
a plurality of needles which are inserted into the outlets of the needle hub, respectively.

2. The needle of claim 1, wherein a plurality of said outlets and a plurality of said needles are arranged in one row, respectively.

3. The needle of claim 1, wherein a plurality of said outlets and a plurality of needles are arranged in multiple rows and columns, respectively.

4. The needle of claim 1, further comprising an adjusting member which is extended from the needle hub so that a tip of each needle can be exposed while covering an intermediate portion of each needle, respectively.

5. The needle of claim 1, further comprising an adjusting member which is inserted into the needle hub and surrounds an intermediate portion of each needle so that a tip of each needle can be exposed, respectively.

6. The needle of claim 1, wherein said needle hub comprises:
a first body which is connected with the nozzle of the injector with the inlet being formed at an end of the first body, and with an accommodating space, which communicates with the inlet, being formed at the other end of the first body; and
a second body through which a plurality of the outlets pass with the second body being inserted into the accommodating space so that each outlet can communicate with the inlet, respectively.

7. The needle of claim 6, wherein said second body is longitudinally formed so that a tip of each needle being exposed, and with an intermediate of each needle being covered, respectively.

8. The needle of claim 1, further comprising a protection cap which is inserted into the needle hub for surrounding the needle so as to protect the same while not exposing the tip of each needle to the outside.
